(19) United States
(12) Patent Application Publication
(10) Pub. No.: US 2008/0281301 A1
(43) Pub. Date: Nov. 13, 2008

(54) Personal Surgical Center

(76) Inventors: Charles DeBoer, Pasasdena, CA (US); Matthew McCormick, Forest Falls, CA (US); Ralph Kerns, Laguna Niguel, CA (US); Prashant Bhadri, Pico Rivera, CA (US); Lawrence Chong, Seal Beach, CA (US); Mark Humayun, Glendale, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/107,052

(22) Filed: Apr. 21, 2008

Related U.S. Application Data
(60) Provisional application No. 60/925,562, filed on Apr. 20, 2007.

(51) Int. Cl. A61B 17/00

(52) U.S. Cl. 606/1

(57) ABSTRACT

A personal surgical center embodied as a general purpose computer (e.g. laptop) with wireless technology for monitoring the operation of an independent surgical center and/or handheld instruments. The computer tracks procedures in the operating room and instruments used during those procedures, and accounts for billing, supply management, and payment options. The monitoring of the instruments used during the surgery is conducted by the personal surgical center while actual control of the settings of those instruments is via the independent surgical center or via controls included in the instruments themselves. The monitored information is stored in a log file which is then transmitted to a hospital server for generating reports, inventory control, billing, and the like. Other information generated during the procedure (e.g. doctor notes) is also stored in the log file. The personal surgical center may also access the hospital server or local data storage device for retrieving a surgeon's specific surgery parameters, obtaining patient files, and the like.

### CROSS-REFERENCE TO RELATED APPLICATION(S)

This application claims the benefit of U.S. Provisional Application No. 60/925,562 (attorney docket D614-59677), filed on Apr. 20, 2007, the content of which is incorporated herein by reference. This application is also related to U.S. Application entitled “Surgical Pack and Tray” (attorney docket D614-61714), and U.S. Application entitled “Independent Surgical Center” (attorney docket D614-61712), both filed on even date herewith, the content of both of which are incorporated herein by reference.

### BACKGROUND OF THE INVENTION

Surgical consoles that are conventional in the art include the Bausch and Lomb Millennium and the Alcon Accurus. The Millennium and Accurus are vitrectomy consoles where each console is configured into a single surgical rolling unit. Each console includes a computer module and surgical modules, and sits on a cart for portability. The computer module controls and monitors the operating parameters of one or more surgical instruments. For example, the computer module may include various buttons which may be actuated by a scrub nurse for console setup or to change settings on the console. A foot pedal is also incorporated into the console to activate certain surgical instruments, such as aspiration and cut speed. Other surgical instruments such as irrigation devices, and illumination devices are also directly controlled via the console. In this manner, the console acts as the center of control for the different surgical instruments.

One drawback of existing surgical consoles is that they sit or are fully integrated into a cart and are not portable by hand. Instead, the consoles are rolled around the operating room by side handles on the cart, putting a limit to where and how they may be transported, as well as limiting their maneuverability within an operating room. Thus, surgical consoles are traditionally with a sterile barrier with the scrub nurse to change settings. The surgeon relies on the scrub nurse to make these adjustments during surgery.

The level of integration of the surgical instruments into the surgical console also varies. Some systems, such as the Alcon Accurus, may have a light and cutter devices directly integrated into the console. Other systems, such as the Bausch and Lomb Millennium, may contain a number of swappable devices. The number and type of swappable devices, however, are limited. Thus, prior art surgical consoles have limited versatility and upgradeability.

### SUMMARY OF THE INVENTION

Embodiments of the present invention are directed to a next generation device (hereinafter referred to as a personal surgical center) for conducting surgeries and other medical procedures, such as, for example, vitreoretinal and cataract surgeries. Unlike the prior art surgical consoles, the personal surgical center according to embodiments of the present invention is implemented via a general purpose computer such as, for example, a laptop. A laptop has a small or no footprint, freeing up operating room space and allowing the center to be easily stored and maneuvered. The laptop may be accessible and easily used in any type of office setting, surgery center, or operating room. The use of the general-purpose computer also makes the surgical center more affordable and less intimidating to people.

According to one embodiment of the invention, the general-purpose computer is configured with one or more software programs for monitoring different surgical instruments and/or independent surgical center. The software programs are configured to automatically recognize one or more instruments and monitor their status and operational parameters during a surgical procedure. While the monitoring is provided by the personal surgical center, the actual control of the surgical instruments is via an independent surgical center and/or via control and logic in the instruments themselves.

Communication with the surgical instruments and/or independent surgical center is preferably via wireless technology. A wireless system allows for fast and efficient setup of the surgical instruments and/or independent surgical center module, which in turn helps reduce operating procedure time and lowers costs. A wireless environment further allows a surgeon to move around his or her environment easily, and improves maneuverability and precision of the surgical instruments. Furthermore, the wireless environment allows instruments to be easily replaced or swapped depending on the desired surgical procedure.

According to one embodiment of the invention, the personal surgical center includes hardware and software for accessing a data communications network (e.g. a hospital information network). Once connected, the personal surgical center may obtain information stored in a network database, such as, for example, patient or doctor records. Information monitored during a particular procedure may also be transmitted over the data communications network for storing in a central database. Access to the network also allows the personal surgical center to access other programs or tools offered by the network, such as, for example, order entry tools for placing orders of surgical instruments.

According to one embodiment, the present invention is directed to a surgical system that includes a portable surgical platform configured to perform at least one of cutting, resecting, illuminating, lasering, aspirating, infusing, cauterizing, cryoperserving biological tissue and fluids, and infusing and aspirating fluids in a human body during surgical procedures. The surgical platform is at least in part disposable. The surgical system further includes a monitoring center coupled to the surgical platform for monitoring one or more operating parameters during the surgical procedures.

According to one embodiment of the invention, the surgical platform is a programmable electronic platform.

According to one embodiment of the invention, the surgical system further includes a display device for displaying an output and functional status of one or more medical instruments used for performing the surgical procedure, and a data store for recording the output and functional status locally or at a remote site.

According to one embodiment of the invention, the surgical platform is battery driven.

According to one embodiment of the invention, the surgical platform is wireless.

According to one embodiment of the invention, the surgical system further includes prepackaged instruments for performing the surgical procedures.

According to one embodiment of the invention, the surgical platform is integrated with a sterile surgical drape.

According to one embodiment of the invention, a sterile interface mechanism allows a non-sterile person to deliver materials into a surgical field.

According to one embodiment of the invention, the surgical platform is part of a sterile surgical field.

According to one embodiment of the invention, the surgical platform includes instruments to be handheld, at an end of an endoscopic system, or configured to be held by a robot.

According to another embodiment, the present invention is directed to a personal surgical center comprising a portable computer unit in wireless communication with at least one of a plurality of handheld instruments, the portable computer unit including a processor and memory having program instructions stored therein, the processor being operable to execute the program instructions, the program instructions including: automatically identifying at least one of the plurality of handheld instruments; wirelessly receiving operation status of the identified handheld instruments; monitoring changes in the operation status of the identified handheld instruments; and displaying the operation status on a display.

According to one embodiment of the invention, the program instructions further include: automatically identifying a user of the portable computer unit; retrieving preference settings for the identified user; and communicating with a network system for accessing patient records, multimedia, doctor files and inventory status.

According to one embodiment of the invention, the personal surgical center includes a memory device or identification card for recognizing personnel accessing the center.

According to one embodiment of the invention, the program instructions further include automatically recognizing an instrument pack including the handheld instruments; and automatically setting up for a corresponding surgical procedure based on the identification.

According to one embodiment of the invention, the program instructions further include loading surgeon specific multimedia clips.

According to one embodiment of the invention, the program instructions further include tracking surgical times, use of instruments, and case statistics.

According to one embodiment of the invention, the program instructions further include recording physician comments and notes.

According to one embodiment of the invention, the program instructions further include turning on the instruments when an instrument pack holding the instruments is opened.

According to another embodiment of the invention, the present invention is also directed to a surgical system that includes a control system accessible by a surgeon for controlling operational parameters of a one or more medical instruments. The surgical system further includes a monitoring system in wireless communication with the control system. The general purpose computer includes a processor and memory with stored program instructions where the processor is operable to execute the program instructions. The program instructions include: wirelessly identifying the medical instruments controlled by the control system; wirelessly receiving the operational parameters of the one or more medical instruments from the control system; monitoring changes in the operational parameters of the one or more medical instruments; and displaying the operational parameters of the one or more medical instruments on a display coupled to the general purpose computer.

According to one embodiment of the invention, the program instructions further include logging the received operational parameters and changes in the operational parameters in a log file, and generating a report based on the logged information.

According to one embodiment of the invention, the operational parameters include at least one of a cutting rate of a surgical cutter, flow rate of an infusion device, vacuum level of an aspiration device, and intensity of an illumination device. The operation parameter may also be battery status of the one or more medical instruments, and/or may identify faulty operation of the one or more medical instruments.

According to one embodiment of the invention, the program instructions further include generating an alert in response to the monitored changes.

According to one embodiment of the invention, the medical instruments include at least one of a biological tissue cutting device, illumination device, infusion device, and aspiration device.

According to one embodiment of the invention, the control system is included in a surgical tray.

According to one embodiment of the invention, the program instructions for the monitoring include program instructions for changing by the control system at least one operational parameter of at least one of the medical instruments; transmitting the change of the at least one operational parameter by the control system; receiving by the general purpose computer the change in the at least one operational parameter; and logging the change in the log file.

According to one embodiment of the invention, the general purpose computer is a laptop.

According to one embodiment of the invention, the control system is located within a sterile field and the monitoring system is located outside the sterile field.

These and other features, aspects and advantages of the present invention will be more fully understood when considered with respect to the following detailed description, appended claims, and accompanying drawings. Of course, the actual scope of the invention is defined by the appended claims.

### BRIEF DESCRIPTION OF THE DRAWINGS

FIG. 1 is a block diagram of a surgical system according to one embodiment of the invention;

FIG. 2 is a schematic block diagram of a personal surgical center according to one embodiment of the invention; and

FIG. 3 is a flow diagram of a process executed by software loaded into the personal surgical center of FIG. 2 according to one embodiment of the invention.

### DETAILED DESCRIPTION

In general terms, the present invention is directed to a personal surgical center that is embodied in a general purpose computer. In this regard, the general purpose computer includes software which, when invoked, allows the computer to communicate with different control systems and/or surgical instruments. The software or updates to the software to allow this functionality may be provided in a portable storage medium (e.g. CD or DVD) or downloaded over the Internet. As the computer is a general purpose computer, other software and programs may also be run on the computer.

The personal surgical center according to one embodiment of the invention takes the form of a laptop or hand held computing device. The center fits in a small case and is portable by hand. This allows the center to be easily moved from one setting or room to the next. Furthermore, the small size of the center facilitates its storage.

The general purpose computer is equipped with wireless technology which allows the personal surgical center to communicate with different handheld instruments and/or independent surgical center(s) (collectively referenced as instrumentations) in a wireless manner. According to one embodiment of the invention, the computer dictates the interactions of the multiple independent surgical centers, tracks procedures in the operating rooms (or in the surgical center) and instruments used during those procedures, and accounts for billing, supply management, and payment options. According to another embodiment of the invention, multiple portable computer units interact with a central server in a wireless manner. The central server uses information wirelessly gathered from the various portable computer units to interact with a hospital system server.

According to one embodiment of the invention, while the monitoring of the surgery and instruments used during the surgery is conducted by the personal surgical center, actual control of the settings of the various medical instruments is via the independent surgical center or via controls included in the instruments themselves. As described in further detail in U.S. Application entitled “Independent Surgical Center” filed on even date herewith, the independent surgical center is the center of control of at least some of the medical instruments used during surgery. According to one embodiment of the invention, the independent surgical center (also referred to as a control system) is configured to be located within the sterile surgical field within which the surgeon operates, and thus, is capable of being manipulated by the surgeon himself during surgery. According to one embodiment of the invention, the sterile barrier consists of a polymer film with gloves incorporated that the nurse can put his/her hands into to assist in the surgical field.

The personal surgical center, on the other hand, is configured to be located outside the sterile barrier and provides other functions including the monitoring and logging of the operating parameters of the various instrumentations during surgery. In this manner, the control of the various instrumentations is kept separate from the monitoring of such instrumentations. Thus, should the personal surgical center become non-operational, the various instrumentations still remain operational.

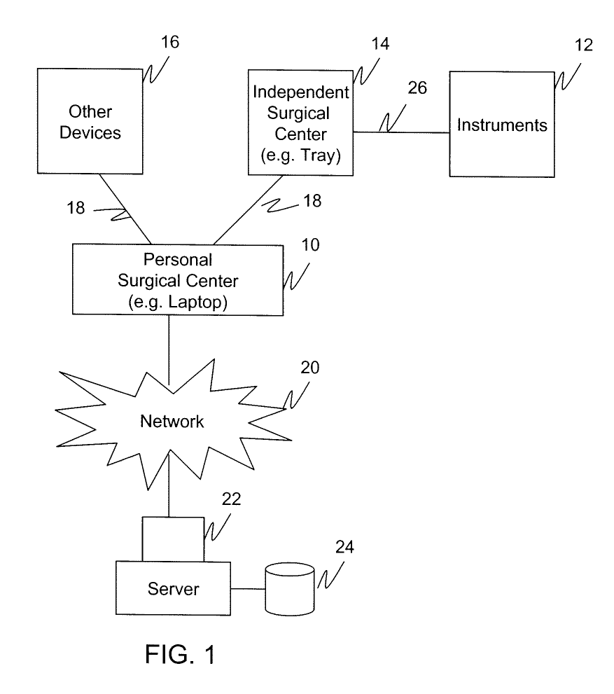

FIG. 1 is a block diagram of a surgical system according to one embodiment of the invention. The system includes a personal surgical center 10 (also referred to as a monitoring center) operably coupled to an independent surgical center 14 and/or other devices 16, preferably over a wireless connection 18. The wireless connection 18 may be, without limitation, a wireless local area connection, such as, for example, an 802.11 connection, a personal area network connection such as, for example, Bluetooth, or any other radio or cellular connection conventional in the art. Although a wireless connection is preferred, a person of skill in the art should recognize that wired connections (e.g. fire wire, parallel port, USB, or other connection) are also possible in addition or in lieu of the wireless connection.

The independent surgical center 14 may take the form of any programmable control system that is located within a surgical tray or handheld devices, such as, other medical instruments 12. The independent surgical center controls these other medical instruments over a data communications link 26. The data communications link 26 may be a wireless connection, wired connection, or a combination of both. According to one embodiment of the invention, the independent surgical center and associated medical instruments 12 provide a portable, in part disposable, surgical platform for use anywhere in the human body that is able to cut, resect, illuminate, laser, aspirate, infuse, cauterize, cryoperserve biological tissue and fluids, and infuse/aspirate sterile fluids used for irrigation during surgical procedures. The independent surgical center may be programmed with specific instrument settings for controlling one or more instruments according to those settings.

The personal surgical center 10 may take the form of any portable, general purpose computer conventional in the art. Although a laptop is preferred in one embodiment, a person of skill in the art should recognize that the personal surgical center 10 may also be a hand held computer, desktop, or any other computing/monitoring device conventional in the art.

The other devices 16 coupled to the personal surgical center 10 include, but are not limited to, secondary display screens, audio outputs, and the like. For example, the personal surgical center may be coupled to a widescreen monitor for displaying the surgical settings in a manner that is overlaid with video of images captured from a microscope or other viewing instrument.

The medical instruments 12 include but are not limited to cutters, aspiration devices, irrigation devices, viewing devices, illumination devices, and/or the like. According to one embodiment of the invention, the independent surgical center and/or instruments 12 are contained in procedure specific surgical packs. For example, an exemplary surgical pack may contain a biological tissue cutting and fluid aspiration system, a biological tissue illuminator, an aspiration and infusion cassette, and other disposable instrumentation. Such other disposable instrumentation may include a disposable speculum/drape combination, syringe for local anesthesia, air/fluid exchange device, syringe for oil exchange, syringe for Triamcinolone steroid, disposable forceps, q-tips, beta-iodine for sterilization, small sterile container with balanced salt solution (BSS), and trocars with pre-mounted cannulas. The surgical packs may be adaptable and customizable for specific surgeons. Furthermore, the instrument holders within the surgical pack may be illuminated via LEDs and the like for identifying the instruments held by the pack. The instruments in the pack may be handled, at the end of an endoscopic system, or configured to be held by a robot. According to one embodiment of the invention, the pack also includes a switch, button, or other mechanism on the pack and/or instrumentation for turning on the instrumentation contained in the pack when the pack is opened. The actuating of the switch or button to turn on the instrumentation may be manual or automatic based on sensing of removal of the instrumentation from the pack.

According to one embodiment of the invention, the personal surgical center 10 is coupled to a system server 22 over a wired or wireless data communications network 20 such as, for example, a local area network, private wide area network, or the public Internet. The system server 22 is in turn coupled to a mass storage device 24 centrally storing information for access by different personal surgical centers over the data communications network. For example, the system server 22 may be a hospital system server storing doctor records, patient records, instrumentation records, scheduling information, and the like, in the mass storage device 24. The mass storage device may store one or more log files generated by the personal surgical center which contains information of the instruments monitored during one or more surgeries.

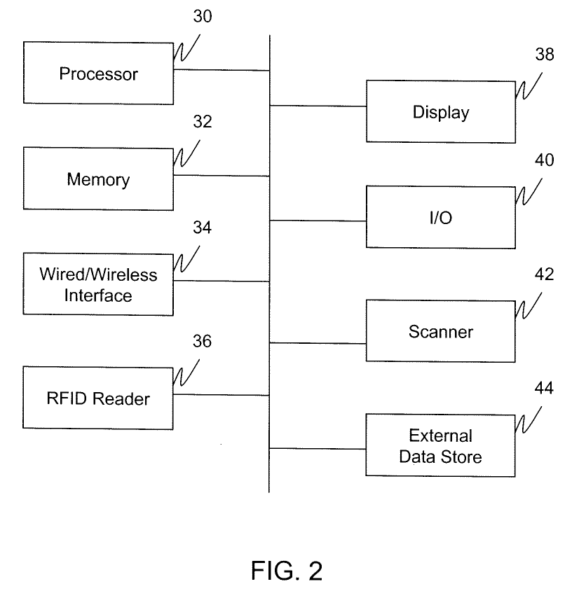

FIG. 2 is a schematic block diagram of the personal surgical center 10 according to one embodiment of the invention. As any general purpose computer, the personal surgical center includes a memory 32 and processor 30 for storing and running different types of software including software that communicates with the independent surgical center(s) 14 and/or other devices 16 for monitoring their operation during a surgical procedure. The same (or separate) software enables the personal surgical center to connect to the system server 22 to access patient records and billing information.

According to one embodiment of the invention, the same (or separate) software also tracks users of the personal surgical center, the room in which the center is being used, the instrumentations being used, the type of procedure being performed, and the timing of such procedure. The software may interface with a master schedule maintained by the system server 22 to coordinate surgeon and operating room schedules, and dynamically change the schedules if required. The system server may also transmit notifications to staff or patients of expected delays (and the expected time of delay) as well as any changes to the schedule.

According to one embodiment of the invention, the software tracks the use of medical instruments 12, to prevent them from being reused. The tracked information may be submitted to the system server 22 for updating its inventory list as well as for billing the patient for the instrument that was used. According to one embodiment of the invention, the inventory list is monitored (either locally by the personal surgical center or centrally by the system server), for allowing instruments with a low stock to be automatically reordered. A report may also be generated when stock is near expiration.

According to one embodiment of the invention, the personal surgical center 10 interacts with an external storage device 44 such as a USB key, CD, DVD, Blue-Ray, HD-DVD, or hard drive for reading and/or writing information from and/or to the storage device. For example, the storage device may be used for performing software upgrades, for local storage of information gathered before, during, and after a surgical procedure (e.g. Optical Coherence Tomography information, notes, patient records, video, pictures, surgeon's comments, etc.), and for storing surgeon information including specific settings to be used during the surgical procedure (e.g. cutting rate of a surgical cutter, flow rate and infusion pressure of an infusion device, vacuum level of an aspiration device, light intensity of an illuminator, and the like). Other setting information may include, for example, screen settings, display settings, chair settings, table settings, room lights and other light settings, thermostat, music, camera settings, microphone settings, and the like. Of course, all this information may also be uploaded and/or downloaded to and/or from the system server 22. If stored in a portable storage medium, the data may be protected via encryption and/or authentication mechanisms conventional in the art.

The personal surgical center 10 is further equipped with a wired and/or wireless interface 34 conventional in the art for wired and/or wireless communication with the server 22 and/or other devices. For example, communication with the server 22 may occur before a surgical procedure to obtain patient records, surgeon's preferred settings, and the like. The personal surgical center 10 may also transmit to the server 22 the instruments that are detected by the center as being present in the room, as well as information of instruments manually entered by a user. Information on the surgeon and surgical staff present in the room may also be transmitted to the server.

An input/output device 40 allows the input/output of information to/from the personal surgical center 10. Exemplary input devices include, but are not limited to a keyboard, mouse, stylus, microphone, camera, or the like. For example, patient records may be retrieved by a surgeon or nurse before a procedure based on specific patient information entered via the keyboard. A wireless microphone may be used during the surgical procedure to enter notes and other surgeon comments. A video camera may also be utilized to capture video during the surgical procedure.

Voice recognition software may be installed in the personal surgical center to receive and process voice commands entered via the wireless microphone. For example, voice commands may be used to control the surgical settings. Input may further be provided via touch-tone technology which allows a user to enter data by merely touching the screen. For example, a user may touch the screen to set the initial operating parameters. The personal surgical center may also be coupled to a screen and/or other output devices for displaying and/or otherwise outputting information before, during, and after a procedure, such as, for example, patient records, instrument settings, and the like.

According to one embodiment of the invention, the personal surgical center 10 is also equipped with a scanning device 42 such as a card reader or sensor for receiving different types of identification information. For example, the scanning device 42 may be used to scan a bar code on a surgical pack or other medical instrument to identify the contents of the pack or the medical instrument. In another example, the scanning device 42 may be used to recognize the surgeon and other personnel in an operating room based upon information stored, for example, in their ID badges. Alternatively, the scanning device may be a biometric scanner for identifying the surgeon and personnel based on their fingerprints, retinal scans, or other biometric information. Recognized staff information may be transmitted to the system server 22 for updating staff location information and/or updating a master schedule. According to one embodiment of the invention, information stored in the central server 22 that is associated with the surgeon may be retrieved upon the automatic recognition of the surgeon. Such retrieved information may include, for example, preferred instrument settings, to allow the center to start with the correct settings automatically. Such setting information may include, for example, a preferred flow rate and pressure of an infusion device, vacuum level of an aspiration device, cutting rate of a surgical cutter, light intensity of an illuminator, or the like. Other settings are also envisioned depending on the type of surgical procedure to be performed. The setting information may also be stored locally in the storage device 44 (e.g. a CD).

According to one embodiment of the invention, the personal surgical center 10 is equipped with an RFID reader 36 which may be any RFID reader conventional in the art. The RFID reader is configured to wirelessly obtain information from the various instrumentations that may be present during surgery. Such information may include, for example, a device ID, expiration date, order number, and the like. Once identified, the personal surgical center monitors the various instrumentations during the surgery. The monitored information may include, for example, a particular instrumentation's operating parameters, battery life, alerts, fault information, and the like. Information on the various instrumentations used during a particular surgery may then be stored locally in the data store 44, or centrally in the server 22 in association with the specific surgery and/or patient. Reports, billing statements, inventory information may then be generated based on the gathered information.

According to one embodiment of the invention, the instrumentations monitored by the personal surgical center are wireless. These wireless instruments contain the circuitry, power, and logic to drive and control themselves. For example, surgical controls (knobs, switches, slides, etc.) are integrated into the instrumentations and manipulated by a user to control operation of different instruments. The wireless instrumentations are also configured to wirelessly transmit identification, status, and operating parameter information to the personal surgical center.

According to one embodiment of the invention, the wireless instrumentations are equipped with an RFID tag. The RFID tag may be passive, semi-passive, or active. If equipped with a passive RFID tag, the tag has no internal power supply and is instead powered when interrogated by the RFID reader 36. If equipped with an active RFID tag, the tag has its own internal power supply and is therefore capable of broadcasting its information to the RFID reader. According to one embodiment of the invention, in addition to the identification information, the RFID tag may also communicate other information stored for the instrumentation, such as, for example, status information, current operating parameters, and the like.

An exemplary self-contained handheld instrument monitored by the personal surgical center 10 is a light pipe. The light pipe may use LED illumination technology such as the one described in U.S. Provisional Application Ser. No. 60/858,176, entitled “Opthalmic Illumination System,” filed on Nov. 10, 2006, the content of which is incorporated herein by reference. Otherwise, the light pipe may be wired directly to the independent surgical center. According to one embodiment of the invention, the light pipe integrates intensity and tinting controls for allowing a user to control the light intensity and tinting directly from the light pipe.

Another exemplary self-contained handheld instrument monitored by the personal surgical center 10 is a biological tissue cutting and fluid aspiration system. The system may be a battery-powered unit, which communicates wirelessly with the personal surgical center and/or interacts with an aspiration/infusion cassette. Cut speed and aspiration may be controlled via controls on the handpiece or from a wireless foot switch.

According to one embodiment of the invention, the handheld instruments are disposable, with the batteries and PCB boards being recyclable. Disposal may include shipping the instrumentation to a specific disposal site. The instruments may be broken down at the disposal site. Components like batteries may be tested, recharged, and re-used immediately, or recycled.

According to one embodiment of the invention, instrumentation that does not directly communicate with the personal surgical center (e.g. a phacoemulsification handpiece) may nonetheless communicate with the personal surgical center via the independent surgical center 14. In this regard, instruments such as phacoemulsification and lens fragmentation handpieces are directly wired to the independent surgical center unit. According to another embodiment, one or more of instruments are coupled to the independent surgical center via a wireless link.

The size of the independent surgical center 14 is configured to be minimal to reduce overall weight and size, making it portable. As described in further detail in U.S. Application entitled “Independent Surgical Center” filed on even date herewith, the independent surgical center houses different drive components for different medical instruments that do not directly communicate with the personal surgical center. For example, the independent surgical center may include a light source, power or pneumatic air for a biological tissue cutting and fluid aspiration system, aspiration and/or pressure for surgical instrumentation, an aspiration cassette, power for cautery, and pressure for an infusion container. For example, infusion may be provided by a pressurized infusion container. The pressurized infusion container may be powered by a small disposable pump, that is located on the independent surgical center.

According to one embodiment of the invention, the independent surgical center is directly integrated into the personal surgical center. According to another embodiment of the invention, the independent surgical center is remotely situated and communicates with the personal surgical center in a wireless manner. In the remote embodiment, the independent surgical center may be embodied as a control system located next to the patient's gurney. For additional details refer to above-referenced U.S. Provisional Application entitled “Surgical Pack and Tray,” filed on even date herewith.

The personal surgical center 10 may be used in office settings or economically disadvantaged environments where there are no scrub nurses. In order to allow non-sterile personnel that have access to the personal surgical center 10 to work on sterile components, such as, for example, the independent surgical center 14, a sterile barrier is provided for the surgical pack and tray which may embody the independent surgical center 14.

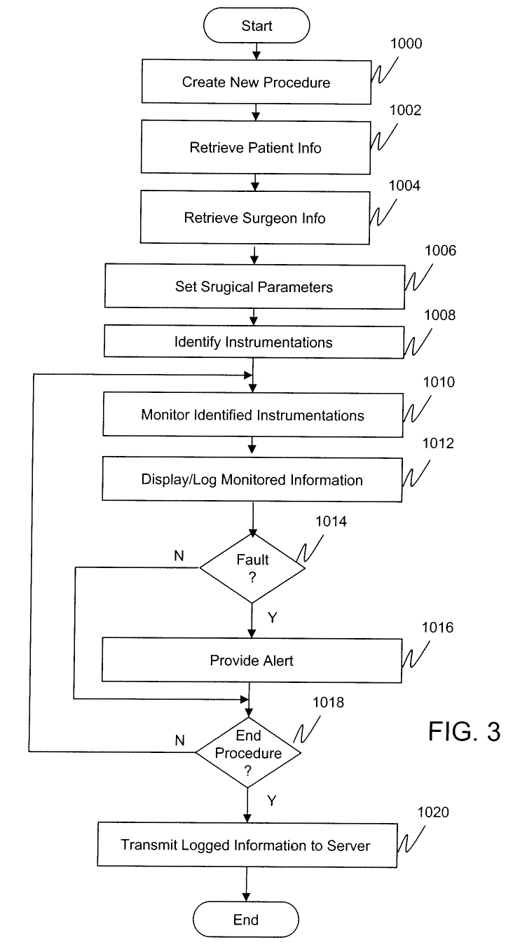

FIG. 3 is a flow diagram of a process executed by software loaded into the personal surgical center 10 according to one embodiment of the invention. The software may be implemented as computer program instructions stored in memory 32 and executed by the processor 30 to cause the processor to engage in the steps illustrated in FIG. 3. A person of skill in the art should understand that the various steps illustrated in this figure may be executed in the order that is shown, or in any other order conventional in the art.

In step 1000, the software creates a new surgical procedure in response to a command by, for example, nurse/doctor. In this regard, the software provides a graphical user interface which allows the nurse/doctor to indicate that the new surgical procedure is to be created, and further allows automatic or manual entry of information associated with the procedure. The entered information is stored in a log file generated for the specific surgical procedure and maintained in the mass storage device 24 and/or locally in the data store 44.

In step 1002, the software retrieves patient information from a patient record stored in the mass storage device 24 and/or data store 44. The patient record may be identified and retrieved based on a patient ID. The retrieved patient information may include, for example, the patient's profile as well as notes, videos, pictures, or other pre-operation information gathered in preparation of the surgical procedure.

In step 1004, the software identifies the surgeon and other personnel to be involved in the surgical procedure as well as the location (e.g. an operating room number) of the personnel. The personnel may be identified, for example, upon interrogation of a user identification card or tag carried by the personnel. The card/tag may store, at a minimum, a user indicia for identifying a user of the card/tag. The user indicia may be retrieved by the scanning device 42 or RFID reader 36 coupled to the personal surgical center, and transmitted to the system server 12 over the data communications network 20. Upon receipt of the user indicia, the system server may invoke a search and retrieval routine for retrieving a personnel record matching the received user indicia.

Upon identification of the surgeon, the software retrieves any preferred or default surgical parameters that may be stored in the surgeon's personnel record. These parameters may include, for example, the preferred or default flow rate and pressure of an infusion device, vacuum level of an aspiration device, cutting rate of a surgical cutter, light intensity of an illuminator, or any other surgical parameters as will be appreciated by a person of skill in the art.

In step 1006, any retrieved preferred and/or parameters are set as the parameters of the current surgical procedure. Any required parameters that are not automatically set are manually entered. The scrub nurse, surgeon, and/or other medical personnel may also modify any of the entered settings during (or before) the surgery as needed.

In step 1008, the software identifies the instrumentations (e.g. handheld instruments and independent surgical centers) that are active in the surgery room, and generates a list of such instrumentations for monitoring. Information on the identified instrumentation as well as other related information, such as, for example, a timestamp of when the instrumentations were recognized, are logged in the log file generated for the particular surgery procedure.

According to one embodiment of the invention, the identification of the instrumentations may be in response to interrogation of bar code data or an RFID tag attached to a surgical pack. The bar code or RFID may also be placed on the packaging of individual instruments or on independent surgical centers. The interrogation may be manually invoked by actuating the RFID reader 36 or scanning device 42 to interrogate an RFID tag or bar code attached to the instrumentations.

Alternatively, the software may be configured to automatically invoke the RFID reader 36 or other sensor to automatically interrogate all instrumentations within its interrogation area. In this regard, the sensor may be configured to automatically transmit a radio signal and identify all responding instrumentations for determining the instrumentations that are present and active in the operating room. The active instrumentations within the interrogation area may respond with their identification information such as, for example, a device ID, name, and model. The software may use the device ID to determine if the instrumentations were previously used. If previously used, an alert may be provided for preventing reuse of the particular instrumentation.

According to one embodiment of the invention, the RFID reader 36 wirelessly transmits a radio signal to the independent surgical center 14. The independent surgical center 14 responds by transmitting its own radio signal containing information about the medical instruments it controls, and the software proceeds to identify the medical instruments 12 based on this response. For example, the response may include the identification information, configuration information, and/or operating parameters of each of the medical instruments 12 that is controlled by the independent surgical center 14. Alternatively, the response may include a device ID, and identification of the actual medical instruments may be obtained by examining the local data store 44 and/or mass storage device 24 and identifying medical instruments that are associated with the device ID. Once the instrumentations are identified, the software is configured to automatically set up for a corresponding surgical procedure based on the identified instruments.

According to an alternative embodiment, a wireless data communications link, such as, for example, a wireless local area network connection may be established between the personal surgical center and the independent surgical center. In this regard, the independent surgical center is equipped with the necessary hardware and software to allow such communication to occur. The data communications link may be created before a surgery and continue until the surgery is over. The data communications link is used by the independent surgical center to transmit identification information, status information, and other monitoring information. In alternative embodiment, the data communications link may also be used to transmit configuration parameters for one or more of the instruments 12. For example, a maximum and minimum light intensity level may be transmitted to an illumination device to configure the illumination device with such maximum and minimum light intensity levels. In yet other alternative embodiments, the wireless communication is between the personal surgical center 10 and one or more surgical instruments. For example, if there are any independent instruments that are not controlled via the independent surgical center 14, the personal surgical center 10 is configured to communicate with those independent instruments separately.

In step 1010, the software obtains from the instrumentations the initial status, settings, and operational parameters of the various instrumentations, and further monitors the instrumentations for any changes in the status, settings, and operational parameters. For example, the software may monitor the battery life, faults, current performance, current operating status of the instruments (e.g. current cut speed, aspiration pressure, and light intensity), and the like. A person of skilled in the art should recognize that the particular operating parameters that are monitored will depend on the particular surgery being performed. In this regard, the center is configured to periodically interrogate all identified instrumentations. The instrumentations respond to radio signals from the RFID reader by transmitting information on their status, settings, and operational parameters. The received information is processed and displayed on the personal surgical center and/or a secondary display device coupled to the center. The received information is also logged in the log file generated for the particular procedure. According to one embodiment of the invention, if any of the identified instrumentations do not respond within a predetermined amount of time, a fault is deemed to have occurred with that instrumentation, and an alert may be provided.

Alternatively, the instrumentations may be programmed to transmit the monitored information to the personal surgical center without a specific request from the personal surgical center. For example, the instrumentations may be configured to automatically transmit the monitored information on a periodic basis. The receiving software may be configured to log only the data that has changed since a prior transmission in the generated log file. The instrumentations may alternatively be configured to transmit the monitored information upon detection of a change of such information. A change may be triggered, for example, when the surgeon changes a particular operational parameter (e.g. cutting rate). The changes are recorded in the log file along with a timestamp in which such changes were recorded.

According to one embodiment of the invention, the software further monitors for new instrumentations introduced before or during a particular procedure. The new instrumentation may be detected, for example, based on an RFID tag or bar code associated with the new instrumentation. The newly detected instrumentation is then added to the list of instrumentations monitored by the software. On a similar note, the software also monitors for instrumentations that have been disabled or removed from the room. Such instrumentations are removed from the list of instrumentations monitored by the software. A timestamp in which the instrumentations have been removed may also be recorded in the log file for the removed instrumentations.

In step 1012, the software displays the information monitored for each instrumentation. The information may be displayed on the personal surgical center or a secondary display monitor coupled to the personal surgical center. For example, the name, model, settings, and operational parameters of each instrument may be displayed on the display along with other monitored information.

In step 1014, a determination is made as to whether the monitored information indicates an operational fault or other status that is identified as being worthy of an alert (e.g. a low battery indication). If the answer is YES, an alert is provided by the software in step 1016. The alert may be an audio alert, visual alert, or a combination of both.

In step 1018, a determination is made as to whether the surgical procedure has ended. The various instrumentations are monitored until a current procedure comes to an end. Once the procedure ends, a timestamp identifying the end is stored in the log file.

In step 1020, the log file is transmitted to the system server 22 for centrally storing in the mass storage device 24. The stored information may then be used for generating statistical information, patient billing, inventory update, and the like. Alternatively, the monitored information is transmitted to the system server 22 in real time as it is being acquired by the personal surgical module.

Hence a surgical system is provided where the monitoring of the surgical instrumentations is separated from the control of such instrumentations. According to one embodiment of the invention, the personal surgical center provides the monitoring functions outside the sterile field whereas the independent surgical center controls one or more medical instruments from within the sterile field. The personal surgical center may be embodied as a portable computing device in wireless (or wired) communication with one or more independent surgical centers and/or medical instrumentations. Specifically, the personal surgical center may be configured for Bluetooth or other wireless communication with instrumentation, the hospital network, cell phones, and other wireless devices. Alternatively, the personal surgical center may not be portable, with only the instrumentations that it monitors being portable.

The use of a general purpose computer as the personal surgical center which is not pre-configured for a certain set of instrumentations but which rather dynamically recognizes the instrumentations that it is to monitor allow the different instrumentations to be freely upgraded, replaced, or mixed and matched as needed or desired. Furthermore, the personal surgical center itself can be freely updated by updating its software via downloads over the Internet or via updates stored in portable media (e.g. CD, DVD, Blu-Ray, HD-DVD, USB drive, or portable hard drive).

According to one embodiment of the invention, the personal surgical center is coupled to the system server for accessing patient records, multimedia clips, and personnel files. This information may also be stored in a portable hard drive(s) accessible by the personal surgical center. This information may be uploaded from the server or portable hard drive for a particular surgical procedure.

The monitoring performed by the personal surgical center includes monitoring instrument settings, battery power, surgery times, use of instruments, and case statistics. According to one embodiment of the invention, low battery may cause the center to issue a warning to the surgeon and staff. The monitoring information is also used to track current stock and automatically re-order instruments when necessary and plan based on scheduled procedures.

The monitoring may further include monitoring the personnel involved in the surgical procedure for outputting staff location to a main computer, a whiteboard, or other device. The monitored information may also be used to re-scheduling operating rooms as required. Information on a recognized surgeon may further be used to retrieve the surgeon's specific surgery parameters, load surgeon specific multimedia clips, and the like.

According to one embodiment of the invention, the personal surgery center records physician comments and notes provided before, during, and after a surgery procedure.

A person of skilled in the art should recognize, therefore, that there are several advantages to the surgical center described in accordance with the above embodiments.

1. low cost: The surgical system according to the above embodiments is a PC based system with most of the system functionality in the surgical tray and instruments themselves; and therefore, has a low cost of goods. These features will encourage surgeons in the United States as well as the global market to begin using this system. It also allows the system to break into new markets including the impoverished nations, other international markets, as well as surgery centers and the doctor's office.

2. Wireless: The surgical system according to the above embodiments includes wireless capabilities for fast and efficient setup at the beginning of the procedure. This in turn reduces operating procedure time and lowers the cost of the procedure. The wireless capabilities also allow the surgeon to move around the operating room/surgery center/office easily. A wireless foot pedal reduces setup time and clutter under the operating table. A wireless illuminator lowers torque on the surgeon's hand, improving maneuverability and precision.

3. “Laptop” based: The personal surgery center according to certain embodiments runs on a “laptop” type computer which reduces instrumentation cost. A “laptop” environment is familiar to the surgical staff and helps reduce the learning curve. Furthermore, upgrading will be easier. The “laptop” environment will allow each hospital system to install and run its own programs. This may help in accessing patient files before and after the procedure.

4. Network capability: The system will be able to log on to the network at the hospital. This will allow the surgical staff to access hospital records, connect to the order entry system, or connect with the main desk.

5. Touch screen monitor: The “laptop” system will have the capability to output to a large surgical flat screen monitor. This will display the current surgical parameters as well as possibly overlay them onto surgical video currently being captured. Touch screen capability may be integrated into the monitor.

6. Surgeon specific patient information: According to one embodiment, each surgeon will have his/her own laptop or a disk with surgeon specific information to be loaded to a shared laptop. The “laptop” is configured to record and save video from the procedure, record surgical notes from the case, display patient specific information (such as preclinical diagnosis, and any other information), and may record procedure time, the length of time instruments are used, and other surgical parameters.

Although this invention has been described in certain specific embodiments, those skilled in the art will have no difficulty devising variations to the described embodiment, which in no way departs from the scope and spirit of the present invention. For example, although the above embodiments contemplate monitoring of the instruments via the independent surgical center 14, a person of skill in the art should recognize that the personal surgical center may also monitor the instruments directly via wireless communication. For example, this may occur in embodiments where no independent surgical center is employed. Furthermore, to those skilled in the various arts, the invention itself herein will suggest solutions to other tasks and adaptations for other applications. It is the Applicants' intention to cover all such uses of the invention and those changes and modifications which could be made to the embodiments of the invention herein chosen for the purpose of disclosure without departing from the spirit and scope of the invention. Thus, the present embodiments of the invention should be considered in all respects as illustrative and not restrictive.

What is claimed is:

1. A surgical system comprising:
a portable surgical platform configured to perform at least one of cutting, resecting, illuminating, lasering, aspirating, infusing, cauterizing, cryoperserving biological tissue and fluids, and infusing and aspirating fluids in a human body during surgical procedures, wherein the surgical platform is at least in part disposable; and
a monitoring center coupled to the surgical platform for monitoring one or more operating parameters during the surgical procedures.

2. The surgical system of claim 1, wherein the surgical platform is a programmable electronic platform.

3. The surgical system of claim 1 further comprising:
a display device for displaying an output and functional status of one or more medical instruments used for performing the surgical procedure; and
a data store for recording the output and functional status locally or at a remote site.

4. The surgical system of claim 1, wherein the surgical platform is battery driven.

5. The surgical system of claim 1, wherein the surgical platform is wireless.

6. The surgical system of claim 1 further comprising prepackaged instruments for performing the surgical procedures.

7. The surgical system of claim 1, wherein the surgical platform is integrated with a sterile surgical drape.

8. The surgical system of claim 1 further comprising a sterile interface mechanism to allow a non-sterile person to deliver materials into a surgical field.

9. The surgical system of claim 1, wherein the surgical platform is part of a sterile surgical field.

10. The surgical system of claim 1, wherein the surgical platform includes instruments to be handheld, at an end of an endoscopic system, or configured to be held by a robot.

11. A personal surgical center comprising a portable computer unit in wireless communication with at least one of a plurality of handheld instruments, the portable computer unit including a processor and memory having program instructions stored therein, the processor being operable to execute the program instructions, the program instructions including:
automatically identifying at least one of the plurality of handheld instruments;
wirelessly receiving operation status of the identified handheld instruments;
monitoring changes in the operation status of the identified handheld instruments; and
displaying the operation status on a display.

12. The personal surgical center of claim 11, wherein the program instructions further include:
automatically identifying a user of the portable computer unit;
retrieving preference settings for the identified user; and
communicating with a network system for accessing patient records, multimedia, doctor files and inventory status.

13. The personal surgical center of claim 11 further comprising:
a memory device or identification card for recognizing personnel accessing the center.

14. The personal surgical center of claim 11, wherein the program instructions further include:
automatically recognizing an instrument pack including the handheld instruments; and
automatically setting up for a corresponding surgical procedure based on the identification.

15. The personal surgical center of claim 11, wherein the program instructions further include loading surgeon specific multimedia clips.

16. The personal surgical center of claim 11, wherein the program instructions further include tracking surgical times, use of instruments, and case statistics.

17. The personal surgical center of claim 11, wherein the program instructions further include recording physician comments and notes.

18. The personal surgical center of claim 11, wherein the program instructions further include turning on the instruments when an instrument pack holding the instruments is opened.

19. A surgical system comprising:
a control system accessible by a surgeon for controlling operational parameters of a one or more medical instruments;
a monitoring system including a general purpose computer in wireless communication with the control system, the general purpose computer including a processor and memory having program instructions stored therein, the processor being operable to execute the program instructions, the program instructions including:
wirelessly identifying the medical instruments controlled by the control system;
wirelessly receiving the operational parameters of the one or more medical instruments from the control system;
monitoring changes in the operational parameters of the one or more medical instruments; and
displaying the operational parameters of the one or more medical instruments on a display coupled to the general purpose computer.

20. The surgical system of claim 19, wherein the program instructions further include:
logging the received operational parameters and changes in the operational parameters in a log file; and
generating a report based on the logged information.

21. The surgical system of claim 19, wherein the operational parameters include at least one of a cutting rate of a surgical cutter, flow rate of an infusion device, vacuum level of an aspiration device, and intensity of an illumination device.

22. The surgical system of claim 19, wherein the operational parameter is battery status of the one or more medical instruments.

23. The surgical system of claim 19, wherein the operational parameter identifies faulty operation of the one or more medical instruments.

24. The surgical system of claim 19, wherein the program instructions further include:
generating an alert in response to the monitored changes.

25. The surgical system of claim 19, wherein the medical instruments include at least one of a biological tissue cutting device, illumination device, infusion device, and aspiration device.

26. The surgical system of claim 19, wherein the control system is included in a surgical tray.

27. The surgical system of claim 19, wherein the program instructions for the monitoring include program instructions for:
changing by the control system at least one operational parameter of at least one of the medical instruments;
transmitting the change of the at least one operational parameter by the control system;
receiving by the general purpose computer the change in the at least one operational parameter; and
logging the change in the log file.

28. The surgical system of claim 19, wherein the general purpose computer is a laptop.

29. The surgical system of claim 19, wherein the control system is located within a sterile field and the monitoring system is located outside the sterile field.
